(19) United States
(12) Patent Application Publication
(10) Pub. No.: US 2019/0300469 A1
(43) Pub. Date: Oct. 3, 2019

(54) METHODS FOR EXTRACTING HYDROXYCINNAMIC ACIDS FROM LIGNO-CELLULOSE

(71) Applicant: UNIVERSITY OF FLORIDA RESEARCH FOUNDATION, INCORPORATED, GAINESVILLE, FL (US)

(72) Inventors: Stephen A. MILLER, Gainesville, FL (US); Steven SHEN, Gainesville, FL (US)

(21) Appl. No.: 16/374,262

(22) Filed: Apr. 3, 2019

Related U.S. Application Data
(60) Provisional application No. 62/651,844, filed on Apr. 3, 2018.

(51) Int. Cl. C07C 59/62; C07C 59/68; C07C 51/43; B01D 11/02

(52) U.S. Cl. CPC C07C 59/62; C07C 59/68; C08L 97/005; B01D 11/0265; C07C 51/43

(57) ABSTRACT

Methods for isolation of ferulic acid and/or p-coumaric acid from lignin paste that may be a resulting waste product from the bioethanol process using sugarcane bagasse or corn stover.

### CROSS-REFERENCE TO RELATED APPLICATION

This application claims the benefit of U.S. Provisional Patent Application No. 62/651,844, filed Apr. 3, 2018, titled Methods for Extracting Hydroxycinnamic Acids from Ligno-Cellulose, which is incorporated by reference herein in its entirety.

### STATEMENT REGARDING FEDERALLY SPONSORED RESEARCH OR DEVELOPMENT

This invention was made with Government support under 2017-68005-26807 awarded by the United States Department of Agriculture and under CHE-1607263 awarded by the National Science Foundation. The Government has certain rights in this invention.

### BACKGROUND

Biomass from plant materials, including forest, agricultural waste, and crop residues, remains the largest renewable resource. This biomass includes aliphatic carbohydrate based polymers, such as cellulose and hemi-cellulose, and the aromatic polymer lignin. Lignin accounts for about 25 percent of all biomass. Lignin is an integral part of the secondary cell walls of plants. Lignin is a cross-linked macromolecule that fills the gap in the cell walls with cellulose, hemicelluloses and pectin components imparting mechanical strength to the cell wall. It is estimated that 50 MMT of lignin is produced annually. Precursors to lignin includes trans-cinnamic acids, such as caffeic acid (3,4-dihydroxycinnamic acid), ferulic acid (3-methoxy-4-hydroxycinnamic), sinapic acid (3,5-dimethoxy-4-hydroxycinnamic), o-coumaric acid (2-hydroxycinnamic) and p-coumaric acid (4-hydroxycinnamic). (Horbowicz et al., Acta Societatis Botanicorum Poloniae 2011, 80(1), 5-9) Sugarcane bagasse contains about 16 percent lignin per dry weight. (Arni et al. Cienc. Tecnol. Aliment. 2007, 5(4) 271-7)

Extraction of phenolic compounds, such as the hydroxycinnamic acids, hydroxybenzoic acids, and flavonoids, employ solvents where the yield is known to be influenced by extraction time, temperature, solvent-to-sample ratio, the number of repeat extractions, and the solvent type. Optimum recovery of phenolics differs from sample to sample depending on the type of plant and its active compounds. Common extraction solvents include water, acetone, ethyl acetate, alcohols (methanol, ethanol and propanol) and their mixtures. Extracted using conventional extraction procedures, such as by the Soxhlet technique, have some substantial disadvantages, including: use of large volumes of hazardous organic solvents; long extraction times; and interference with and degradation of targeted components due to both internal and external factors such as light, air, high temperatures and enzymatic reactions. To overcome at least some of the short-comings of these techniques, use of ultrasound-assisted extraction (UAE), microwave-assisted extraction (MAE), ultrasound-microwave-assisted extraction (UMAE), supercritical fluid extraction (SFE), sub-critical water extraction (SCWE) and high hydrostatic pressure processing (HHPP) have been used to shorten extraction times and reduce organic solvent consumption. (Khoddami et al., Molecules 2013, 18, 2328-75)

Carbohydrate components have been effectively removed from lignocellulose materials, generally by acid-catalyzed hydrolysis and/or application of enzymes; a lignin paste remains, which is available from the waste streams of cellulosic bioethanol facilities that use, for example, sugarcane bagasse or corn stover. It is of interest to isolate ferulic acid and p-coumaric acid from this abundant source of carbohydrate depleted lignin paste.

### BRIEF SUMMARY

Various embodiments relate to a method for isolating at least one target from a lignin source. The method may include adding the lignin source to a base solution to form a suspension or solution; applying energy to the suspension or solution; adding an acid to the suspension or solution to precipitate lignin solids; separating the lignin solids from the suspension or solution; extracting the target from the suspension or solution with an organic solvent to form an organic solution comprising the target; washing the organic solution with water and/or brine; drying the organic solution; and removing the organic solvent from the organic solution to recover a first product comprising a first quantity of the target. These and other features, aspects, and advantages of various embodiments will become better understood with reference to the following description, figures, and claims.

### BRIEF DESCRIPTION OF THE FIGURES

Many aspects of this disclosure can be better understood with reference to the following FIGURES, in which:

FIG. 1: is an example according to various embodiments, illustrating a scheme for extracting p-coumaric acid from a lignin paste.

It should be understood that the various embodiments are not limited to the examples illustrated in the FIGURES.

### DETAILED DESCRIPTION

### Introduction and Definitions

Various embodiments may be understood more readily by reference to the following detailed description. Unless defined otherwise, all technical and scientific terms used herein have the same meaning as commonly understood by one of ordinary skill in the art to which this disclosure belongs.

As used herein, the term “standard temperature and pressure” generally refers to 20° C. and 1 atmosphere. Standard temperature and pressure may also be referred to as “ambient conditions” or “room temperature.” Unless indicated otherwise, parts are by weight, temperature is in ° C., and pressure is at or near atmospheric. The terms “elevated temperatures” or “high-temperatures” generally refer to temperatures of at least 100° C.

The term “mol percent” or “mole percent” generally refers to the percentage that the moles of a particular component are of the total moles that are in a mixture. The sum of the mole fractions for each component in a solution is equal to 1.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant FIGURE.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

The examples and embodiments described herein are for illustrative purposes only and that various modifications or changes in light thereof will be suggested to persons skilled in the art and are to be included within the spirit and purview of this application. Many variations and modifications may be made to the above-described embodiment(s) of the disclosure without departing substantially from the spirit and principles of the disclosure. All such modifications and variations are intended to be included herein within the scope of this disclosure.

As used herein the term “lignin” generally refers to a class of complex organic polymers that form key structural materials in the support tissues of vascular plants and some algae. Lignins are particularly important in the formation of cell walls, especially in wood and bark, because they lend rigidity and do not rot easily. Chemically, lignins are cross-linked phenolic polymers. Sources of lignin may include but are not limited to wood, sugar cane, bagasse, and corn stover. As used herein, “bagasse” refers to the dry pulpy residue left after the extraction of juice from sugar cane. As used herein, “corn stover” refers to the leaves, stalks, and cobs of maize plants.

General Discussion

Various embodiments relate to the isolation of ferulic acid and p-coumaric acid from carbohydrate depleted lignin paste. This abundant source of lignin paste affords the desired hydroxycinnamic acids. The process involves the base solution promoted extraction of the paste followed by acidification and extraction of the acid solution with an organic solvent. According to various embodiments, the organic solvent may be ethyl acetate. According to various embodiments, the extraction of the paste may be carried out at elevated temperatures. According to various embodiments, the extraction of the paste may be assisted by ultrasound. According to various embodiments, the extraction of the paste may be assisted by microwave radiation.

The isolation of ferulic acid and p-coumaric acid from lignin paste that is a resulting waste product from the bioethanol process using sugarcane bagasse or corn stover allows the conversion of waste to a valuable commodity for uses in medicine and industry applications. Ferulic and p-coumaric acid exhibit anti-cancer, anti-viral, anti-bacterial, antiseptic, antioxidant, analgesic, and anti-inflammatory activities and can be incorporated into one's diet or into drug formulations. They may also be employed as safe food preservatives. Additionally, ferulic acid and p-coumaric acid may even be used as biorenewable monomers to produce degradable packaging thermoplastics.

Various embodiments relate to a method for isolating at least one target from a lignin source. The method may include adding the lignin source to a base solution to form a suspension or solution; applying energy to the suspension or solution; adding an acid to the suspension or solution to precipitate lignin solids; separating the lignin solids from the suspension or solution; extracting the target from the suspension or solution with an organic solvent to form an organic solution comprising the target; washing the organic solution with water and/or brine; drying the organic solution; and removing the organic solvent from the organic solution to recover a first product comprising a first quantity of the target.

According to various embodiments, the lignin source may be selected from a sugar cane bagasse, a corn stover, and a combination thereof. The lignin source may be a powder. As used herein, “powder” refers to a plurality of fine, dry particles, such as may be produced by the grinding, crushing, or disintegration of a solid substance. According to various embodiments, the base solution may be a sodium hydroxide solution. According to various embodiments, the organic solvent may be ethyl acetate. According to various embodiments, the at least one target may be selected from ferulic acid, p-coumaric acid, and a combination thereof.

According to various embodiments, applying energy to the suspension or solution may include heating. For example, the suspension or solution may be heated to a temperature above about 100 degrees Celsius. According to various embodiments, the heating may be performed at a pressure greater than atmospheric pressure. For example, according to various embodiments, the suspension or solution may be heated to a temperature of about 150 degrees Celsius at a pressure of about 20 psi. According to various embodiments, applying energy to the lignin source may include sonicating the lignin source. The sonicating may be performed at room temperature. According to various embodiments, applying energy to the lignin source may include irradiating the lignin source with microwaves while maintaining a temperature below 90 degrees Celsius.

According to various embodiments, the method may further include adding an organic solvent to the lignin solids to form an organic solution comprising the lignin solids, separating the lignin solids from the organic solution; washing the organic solution with water and/or brine; drying the organic solution; and removing the organic solvent from the organic solution to recover a second product comprising a second quantity of the target.

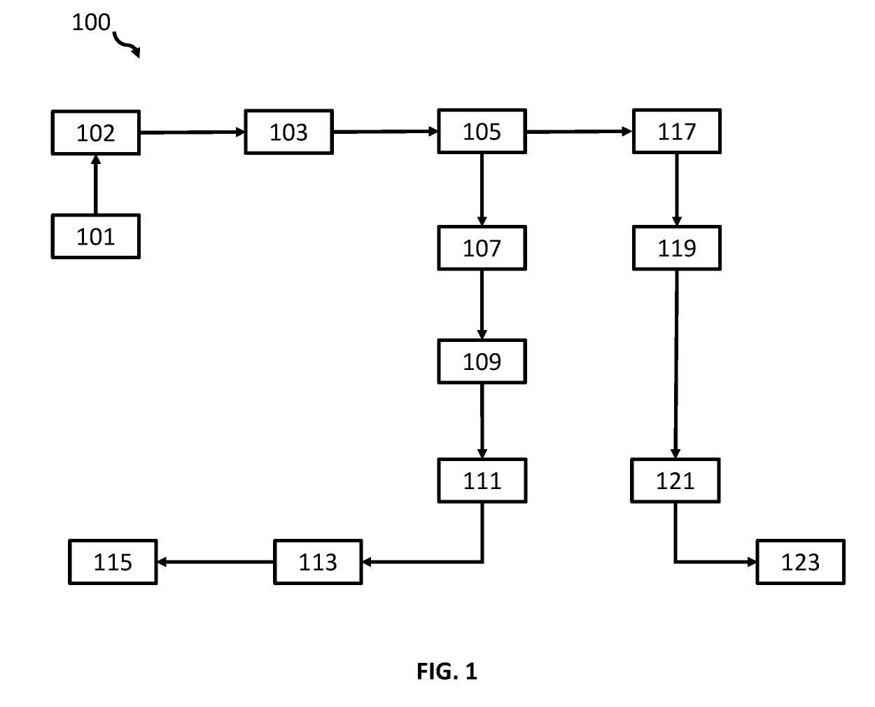

Discussion of FIG. 1

FIG. 1 is an example according to various embodiments, illustrating a scheme 100 for extracting a target compound (for example, p-coumaric acid (pCA) and/or ferulic acid (FA)) from a lignin paste.

At box 101 a lignin powder may optionally be added to a base solution to form a lignin paste. The base solution may include sodium hydroxide or potassium hydroxide. The lignin powder may be present in the lignin paste in an amount within a range having a lower limit and/or an upper limit. The range may include or exclude the lower limit and/or the upper limit. The lower limit and/or upper limit can be selected from about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, and 70 percent by dry weight. For example, according to certain embodiments, the lignin powder may be present in the lignin paste in an amount of about 50 percent by dry weight, or any combination of lower limits and upper limits described. At box 102, energy may be applied to the lignin paste produced at box 101 or as otherwise provided (for example, by a different process or from a different source). Energy may be provided at box 102 in one or more of a variety of ways. According to various embodiments, sufficient energy may be supplied to depolymerize the lignin paste. As discussed above, lignins may include cross-linked phenolic polymers. Depolymerization is the process of converting a polymer into monomers or other smaller units. The degree of depolymerization may vary. For example, according to various embodiments, the degree of depolymerization may be partial or complete.

The energy provided at box 102 may be applied to the lignin paste via heat, sonication, and/or irradiation. For example, at box 102 the lignin paste may be hydrolyzed at a hydrolysis temperature for a time period. The time period may be within a range having a lower limit and/or an upper limit. The range may include or exclude the lower limit and/or the upper limit. The lower limit and/or upper limit can be selected from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 hours. For example, according to certain embodiments, the time period may be about 20 hours, or any combination of lower limits and upper limits described. The lignin paste may be hydrolyzed at a hydrolysis temperature within a range having a lower limit and/or an upper limit. The range may include or exclude the lower limit and/or the upper limit. The lower limit and/or upper limit can be selected from about 50, 55, 60, 65, 70, 75, 80, 85, 90, 95, 100, 105, 110, 115, 120, 125, 130, 135, 140, 145, and 150 degrees Celsius. For example, according to certain embodiments, the lignin paste may be hydrolyzed at a hydrolysis temperature of about 100 degrees Celsius, or any combination of lower limits and upper limits described. After the lignin paste has been hydrolyzed at the hydrolysis temperature for the time period, the resultant hydrolyzed lignin paste may be cooled to a temperature. The temperature may be within a range having a lower limit and/or an upper limit. The range may include or exclude the lower limit and/or the upper limit. The lower limit and/or upper limit can be selected from about 20, 20.5, 21, 21.5, 22, 22.5, 23, 23.5, 24, 24.5, 25, 25.5, 26, 26.5, 27, 27.5, 28, 28.5, 29, 29.5, and 30 degrees Celsius. For example, according to certain embodiments, the temperature may be in a range of from about 20 to about 30 degrees Celsius, or any combination of lower limits and upper limits described. The temperature may be about room temperature.

Optionally, at box 102, the lignin paste may be alternatively or additionally sonicated. The sonication frequency may be within a range having a lower limit and/or an upper limit. The range may include or exclude the lower limit and/or the upper limit. The lower limit and/or upper limit can be selected from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 kHz. For example, according to certain embodiments, the sonication frequency may be about 23 kHz, or any combination of lower limits and upper limits described. As used herein, “sonication” is the act of applying sound energy to agitate particles in a sample, for various purposes such as the extraction of multiple compounds from plants, microalgae and seaweeds. According to various embodiments, the lignin paste may be sonicated for a duration. The duration may be a time period within a range having a lower limit and/or an upper limit. The range may include or exclude the lower limit and/or the upper limit. The lower limit and/or upper limit can be selected from about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 hours. For example, according to certain embodiments, the duration may be a time period of about 2 to about 5 hours, or any combination of lower limits and upper limits described.

Optionally, at box 102, the lignin paste may be alternatively or additionally irradiated, for example with microwaves, for a duration. The microwaves may have a power within a range having a lower limit and/or an upper limit. The range may include or exclude the lower limit and/or the upper limit. The lower limit and/or upper limit can be selected from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, and 70 Watts. For example, according to certain embodiments, the microwaves may have a power of 50 Watts, or any combination of lower limits and upper limits described. The duration may be a time period within a range having a lower limit and/or an upper limit. The range may include or exclude the lower limit and/or the upper limit. The lower limit and/or upper limit can be selected from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, and 45 minutes. For example, according to certain embodiments, the duration may be a time period of about 15 minutes, or any combination of lower limits and upper limits described. During irradiation, the lignin paste may be maintained at a maximum temperature within a range having a lower limit and/or an upper limit. The range may include or exclude the lower limit and/or the upper limit. The lower limit and/or upper limit can be selected from about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 degrees Celsius. For example, according to certain embodiments, the lignin paste may be maintained at a maximum temperature of about 90 degrees Celsius, or any combination of lower limits and upper limits described.

According to various embodiments, applying energy to the lignin paste at box 102 via heat, sonication, and/or irradiation may be accompanied by a pressure increase such that the application of energy is performed at a pressure greater than atmospheric pressure (about 14.696 psi). The pressure increase may be in an amount within a range having a lower limit and/or an upper limit. The range may include or exclude the lower limit and/or the upper limit. The lower limit and/or upper limit can be selected from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 psi. For example, according to certain embodiments, the pressure increase may be in an amount of about 20 psi, or any combination of lower limits and upper limits described. According to various embodiments, applying energy to the lignin paste at box 102 via heat, sonication, and/or irradiation may be performed at a pressure greater than atmospheric pressure. The pressure may be within a range having a lower limit and/or an upper limit. The range may include or exclude the lower limit and/or the upper limit. The lower limit and/or upper limit can be selected from about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55 psi. For example, according to certain embodiments, the pressure may be about 20 psi, or any combination of lower limits and upper limits described.

At step 103, the hydrolyzed, sonicated, and/or irradiated lignin paste (or depolymerized lignin paste) may be acidified, resulting in the precipitation of lignin. The hydrolyzed lignin paste may be acidified to a pH within a range having a lower limit and/or an upper limit. The range may include or exclude the lower limit and/or the upper limit. The lower limit and/or upper limit can be selected from about 0.5, 1, 1.5, and 2. For example, according to certain embodiments, the lignin paste may be acidified to a pH of about 1, or any combination of lower limits and upper limits described. The acidification may be performed by adding a weak acid and/or a strong acid. The weak acid may be any suitable acid, for example, methanoic acid (HCO₂H), acetic acid (CH₃CO₂H), phosphoric acid (H₃PO₄), nitrous acid (HNO₂), or combinations thereof. The strong acid may be any suitable acid, for example, sulfuric acid (H₂SO₄), hydrochloric acid (HCl), nitric acid (HNO₃), hydroiodic acid (HI), or combinations thereof.

The precipitated lignin may be filtered and separated at step 105. The filtration may be performed by any suitable filtration technique, for example by passing the mixture comprising the precipitated lignin through filter paper. The filter paper may have a pore size within a range having a lower limit and/or an upper limit. The range may include or exclude the lower limit and/or the upper limit. The lower limit and/or upper limit can be selected from about 2, 3, 4, 5, 6, 7, 8, 9, 10, 11, 12, 13, 14, 15, 16, 17, 18, 19, 20, 21, 22, 23, 24, and 25 μm. For example, according to certain embodiments, the filter paper may have a pore size of from 10 to 15 μm, or any combination of lower limits and upper limits described. After the filtration and separation step 105, the solids 117 and a target-rich aqueous solution 107 may be processed in different ways.

At step 107, the target compound(s) in the target-rich aqueous solution remaining after filtration 107 may be extracted with a polar, aprotic solvent, such as ethyl acetate, to produce a pCA-rich organic solution 109.

At step 111, the target-rich organic solution 109 may be washed with water, washed with a brine solution, dried using an inorganic salt, such as MgSO₄, filtered, and dried to yield crude target compound (such as, for example, crude pCA, crude FA, or a combination thereof). According to various embodiments, the drying may be performed using rotary evaporation to yield the crude target compound(s) at step 113.

The crude target compound(s) obtained at step 113 may be further purified by recrystallization from water at step 115 to yield purified target compound(s). To accomplish the further purification by recrystallization from water, the crude target compound(s) obtained at step 113 may be dissolved in water and optionally a minimal amount of ethanol. A ratio of about 100 mL water to about 3 to 5 mL ethanol may be used. The volume of the resultant solution may be reduced by about 50% by rotary evaporation. Purified target compound(s) may be precipitated due to the volume reduction. After filtration and drying, high-purity target compound(s) may be obtained.

The solids 117 from the filtration and separation step 105 may be washed with a polar, aprotic solvent, such as ethyl acetate to produce a target-rich organic solution 119.

At step 121, the target-rich organic solution 119 may be washed with water, washed with a brine solution, dried using an inorganic salt, such as MgSO₄, filtered, and dried to yield crude target compound(s). According to various embodiments, the drying may be performed using rotary evaporation to yield the crude target compound(s) at step 123. The crude target compound(s) obtained at step 123 by optionally be further purified by recrystallization from water similarly to step 115 to yield purified target compound(s).

### EXAMPLES

Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for. The purpose of the following examples is not to limit the scope of the various embodiments, but merely to provide examples illustrating specific embodiments.

### Example 1

A purpose of this example is to illustrate an extraction at 100° C. from lignin paste, according to various embodiments.

To 10.00 g of dried lignin paste from sugar cane bagasse, was added 250 mL of 1 M NaOH(aq), and 100 mg of NaHSO₃ in a 500 mL round bottom flask under a nitrogen atmosphere. After stirring at room temperature for 10 minutes, the reaction temperature was raised until the system refluxed. At this point all reagents dissolved to form a dark brown liquid. The reaction was run for 20 hours and cooled to room temperature. To precipitate unreacted lignin, the solution was acidified to pH=1 with concentrated H₃PO₄. The solids were filtered and the remaining solution was extracted with three 100 mL portions of ethyl acetate. The ethyl acetate portion was washed with water, washed with brine solution, and dried using MgSO₄. The ethyl acetate was removed using rotary evaporation to yield crude p-coumaric acid (pCA). The crude pCA was further purified by recrystallization from water by dissolving the pCA in a minimal amount of ethanol and adding 100 mL of water. The volume of the solution was then reduced by about 50% by rotary evaporation from which product precipitated. The remaining solution was cooled and additional product precipitated overnight. High-purity pCA was obtained after filtering and drying to yield 126 mg of pCA. ¹H NMR confirmed the identity and purity of the isolated pCA. Additional pCA was obtained by washing the lignin solids obtained after the acidification step, but this fraction was difficult to purify.

### Example 2

A purpose of this example is to illustrate an iterative extraction at 100° C. from lignin paste, according to various embodiments.

To 2.00 g of dried lignin powder from sugar cane bagasse was added 50 mL of NaOH 0.5 M (aq), in a 100 mL round bottom flask. The suspension was heated for 2 hours at 100° C. The solution was cooled and acidified to pH=1 with 50 mL HCl 1 M (aq). Lignin solids were filtered and washed with deionized water (DIW), and the resulting solution was extracted with 3×100 mL portions of ethyl acetate. The ethyl acetate portion was washed with water, washed with brine solution, and dried using MgSO₄. The ethyl acetate was removed using rotary evaporation to yield crude p-coumaric acid (pCA) and ferulic acid (FA). The identity was confirmed and the purity was measured by ¹H NMR analysis.

The extraction procedure, as given above, was repeated twice using the lignin solids from the previous extraction and two additional extractions were performed on lignin solids with heating for 20 hours and 24 hours. The results are tabulated in Table 1, below.

TABLE 1
Heated at 100° C. Extraction Result

| Extraction No. | Solids Mass (g) | Crude Yield (%) | Ratio pCA:FA |
|---|---|---|---|
| 1 | 0.059 | 2.95 | 88:12 |
| 2 | 0.078 | 3.9 | 84:16 |
| 3 | 0.042 | 2.1 | 70:30 |
| 4^{a} | 0.055 | 2.75 | Trace |
| 5_{b} | 0.041 | 2.1 | Trace |
| TOTAL^{c} | 0.179 | 8.9 | 82:18 |

^{a}Heated for 20 hours,
_{b}Heated for 24 hours,
^{c}total excluding extraction no. 4 and 5

### Example 3

A purpose of this example is to illustrate an iterative Extraction in a Glass Reactor Vessel at 150° C. from lignin paste, according to various embodiments.

To 2.00 g of dried lignin powder from sugarcane bagasse was added 50 mL of NaOH 0.5 M (aq), in a closed 100 mL glass reactor vessel. The suspension was heated for 2 hours at 150° C., resulting in an increase of pressure by 20 psi. The solution was cooled and acidified to pH=1 with 50 mL HCl 1 M (aq). Lignin solids were filtered and washed with deionized water (DIW), and the resulting solution was extracted with 3×100 mL portions of ethyl acetate. The ethyl acetate portion was washed with water, washed with brine solution, and dried using MgSO₄. The ethyl acetate was removed using rotary evaporation to yield crude p-coumaric acid (pCA) and ferulic acid (FA). The identity was confirmed and the purity was measured by ¹H NMR analysis.

The extraction procedure, as given above, was repeated twice using the lignin solids from the previous extraction and two additional extractions were performed on lignin solids with heating for 16 hours and 18 hours. The results are tabulated in Table 2, below.

TABLE 2
Heated at 150° C. Extraction Result

| Extraction No. | Solids Mass (g) | Crude Yield (%) | Ratio pCA:FA |
|---|---|---|---|
| 1 | 0.117 | 5.85 | 83:17 |
| 2 | 0.050 | 2.5 | 74:26 |
| 3 | 0.020 | 1 | 56:44 |
| 4^{a} | 0.028 | 1.4 | 37:63 |
| 5_{b} | 0.048 | 2.4 | 37:63 |
| TOTAL | 0.263 | 13.1 | 66:34 |

^{a}Heated for 16 hours,
_{b}Heated for 18 hours.

### Example 4

A purpose of this example is to illustrate an iterative Ultrasound-assisted Extraction from lignin paste, according to various embodiments.

To 2.00 g of dried lignin powder from sugarcane bagasse was added 50 mL of NaOH 0.5 M (aq) into a 100 mL round-bottom flask. The suspension was sonicated for 2 hours at room temperature. The aqueous solution was acidified to pH=1 with 50 mL HCl 1 M (aq). Lignin Solids were filtered, and washed with DIW. The mother liquor was extracted with three 100 mL portions of ethyl acetate. The ethyl acetate solution was washed with water and brine and dried with anhydrous MgSO₄. The ethyl acetate was removed by rotary evaporation to yield a crude mixture of p-coumaric acid (pCA) and ferulic acid (FA). The identity was confirmed and the purity was measured by ¹H NMR analysis. The lignin solids were loaded in a 100 mL round-bottom flask for an additional sonication cycle employing like conditions for five hours and worked up in a like manner as above. The extraction procedure was repeated as above until no pCA and FA were observed by ¹H NMR analysis. Results are tabulated in Table 3, below.

TABLE 3
Ultrasound Assisted Extraction Result

| Extraction No. | Solids Mass (g) | Crude Yield (%) | Ratio pCA:FA |
|---|---|---|---|
| 1 ^{a} | 0.104 | 5.2 | 80:20 |
| 2 ^{b} | 0.094 | 4.7 | 86:14 |
| 3 ^{b} | 0.066 | 3.3 | 88:12 |
| 4 ^{b} | 0.039 | 1.95 | 83:17 |
| 5 ^{b} | 0.009 | 0.45 | Trace |
| TOTAL^{c} | 0.303 | 15.1 | 84:16 |

^{a} Sonicated for 2 hours,
^{b} Sonicated for 5 hours,
^{c}total excluding extraction no. 5.

### Example 5

A purpose of this example is to illustrate an Iterative Microwave-assisted Extraction from lignin paste, according to various embodiments.

To 2.00 g of dried lignin powder from sugar cane bagasse was added 50 mL of 0.5 M NaOH (aq), in a 100 mL round-bottom flask. The suspension was irradiated with microwaves by a dynamic procedure with a power of 50 Watts and a maximum temperature of 90° C. for 15 minutes. The solution was acidified to pH=1 with 50 mL 1 M HCl (aq). Lignin solids were filtered and washed with DIW, and the combined liquid was extracted with three 100 mL portions of ethyl acetate. The ethyl acetate portion was washed with water and brine and dried over anhydrous MgSO₄. The ethyl acetate was removed by rotary evaporation to yield a crude mixture of p-coumaric acid (pCA) and ferulic acid (FA). The extraction of lignin solids was repeated for four extraction cycles using an identical procedure as above. Results are tabulated in Table 4, below.

TABLE 4
Microwave Assisted Extraction Result

| Extraction No. | Solids Mass (g) | Crude Yield (%) | Ratio pCA:FA |
|---|---|---|---|
| 1 | 0.079 | 3.95 | 86:14 |
| 2 | 0.070 | 3.5 | 84:16 |
| 3 | 0.064 | 3.2 | 84:16 |
| 4 | 0.063 | 3.15 | 76:24^{b} |
| 5 | 0.055 | 2.74 | Trace |
| TOTAL^{a} | 0.276 | 13.8 | 83:17 |

^{a}Total excluding extraction no. 5,
^{b}baseline of spectrum poor.

### Example 6

A purpose of this example is to illustrate an Iterative Extraction in Glass Reactor Vessel at 110° C. from bagasse, according to various embodiments.

To 2.00 g of dried bagasse was added 50 mL of NaOH 0.5 M (aq), in a closed 100 mL glass reactor vessel. The suspension was heated for 2 hours at 110° C., resulting in an increase of pressure by 20 psi. The solution was cooled and acidified to pH=1 with 50 mL 1 M HCl (aq). Solids were filtered and washed with deionized water (DIW), and the resulting solution was extracted with 3×100 mL portions of ethyl acetate. The ethyl acetate portion was washed with water, washed with brine solution, and dried using MgSO₄. The ethyl acetate was removed using rotary evaporation to yield crude p-coumaric acid (pCA) and ferulic acid (FA). The identity was confirmed and the purity was measured by ¹H NMR analysis.

The extraction procedure, as given above, was repeated twice using the bagasse from the previous extraction. The results are tabulated in Table 5, below.

TABLE 5
Heated at 110° C. Extraction Result

| Extraction No. | Solids Mass (g) | Crude Yield (%) | Ratio pCA:FA |
|---|---|---|---|
| 1 | 0.157 | 7.8 | 83:17 |
| 2 | 0.105 | 5.2 | 74:26 |
| 3 | 0.068 | 3.4 | Trace |
| TOTAL^{a} | 0.262 | 13.0 | 79:21 |

^{a}Total excluding extraction no. 3

### Example 7

A purpose of this example is to illustrate an Iterative Ultrasound-assisted Extraction from bagasse, according to various embodiments.

To 2.00 g of dried bagasse was added 50 mL of 0.5 M NaOH (aq), in a 100 mL round-bottom flask. The suspension was sonicated for 2 hours at room temperature. The aqueous solution was acidified to pH=1 with 50 mL 1 M HCl (aq). Solids were filtered, and washed with DIW. The mother liquor was extracted with three 100 mL portions of ethyl acetate. The ethyl acetate solution was washed with water and brine and dried with anhydrous MgSO₄. The ethyl acetate was removed by rotary evaporation to yield a crude mixture of p-coumaric acid (pCA) and ferulic acid (FA). The identity was confirmed and the purity was measured by ¹H NMR analysis. The extraction procedure, as given above, was repeated three times using the bagasse from the previous extraction. The results are tabulated in Table 6, below.

TABLE 6
Ultrasound Assisted Extraction Result

| Extraction No. | Solids Mass (g) | Crude Yield (%) | Ratio pCA:FA |
|---|---|---|---|
| 1 | 0.081 | 4.0 | 67:33 |
| 2 | 0.078 | 3.9 | 74:26 |
| 3 | 0.064 | 3.2 | 100:0^{a} |
| 4 | 0.042 | 2.1 | Trace |
| TOTAL^{b} | 0.223 | 11.1 | 79:21 |

^{a}Sample is too diluted,
^{b}total excluding extraction no. 4.

### Example 8

A purpose of this example is to illustrate an Iterative Microwave-assisted Extraction from bagasse, according to various embodiments.

To 2.00 g of dried lignin bagasse was added 50 mL of 0.5 M NaOH (aq), in a 100 mL round-bottom flask. The suspension was irradiated with microwaves by a dynamic procedure with a power of 50 Watts and a maximum temperature of 90° C. for 15 minutes. The solution was acidified to pH=1 with 50 mL 1 M HCl (aq). Solids were filtered and washed with DIW, and the combined liquid was extracted with three 100 mL portions of ethyl acetate. The ethyl acetate portion was washed with water and brine and dried over anhydrous MgSO₄. The ethyl acetate was removed by rotary evaporation to yield a crude mixture of p-coumaric acid (pCA) and ferulic acid (FA). The extraction of bagasse was repeated for three extraction cycles using an identical procedure as above. Results are tabulated in Table 7, below.

TABLE 7
Microwave Assisted Extraction Result

| Extraction No. | Solids Mass (g) | Crude Yield (%) | Ratio pCA:FA |
|---|---|---|---|
| 1 | 0.054 | 2.7 | 74:26 |
| 2 | 0.076 | 3.8 | 72:28 |
| 3 | 0.055 | 2.75 | 66:34 |
| TOTAL | 0.185 | 9.2 | 71:29 |

Purification using activated carbon

Activated carbon can be used to purify p-coumaric acid (pCA) and ferulic acid (FA). This method is adapted from a paper published by the Ou group (Ou et al., J. Food Eng. 2007, 78, 1298-1304). 140 mg of crude product and 5 mL of acetone were combined in a 100 mL beaker. Then, 25 mL of warm pH 3 HCl (aq) was added to the beaker slowly while heating on a hotplate, resulting a dark-brown solution. The solution was heated for 15 minutes at 80° C. to remove the acetone from the solution. A pea sized portion of activated carbon was added into the solution, and it was stirred for 1 hour at 80° C. The charcoal was filtered out by gravity filtration and washed with pH 3 HCl (aq), resulting a light yellow filtrate. Then, the filtrate was extracted with 2×10 mL portions of ethyl acetate. The organic layer was washed with 15 mL of water, followed by brine, and then dried over anhydrous MgSO₄. The ethyl acetate was then removed by rotary evaporation leaving behind an off white powder. The identity was confirmed and the purity was measured by ¹H NMR analysis. The purification yield was 42.1% with pCA:FA of 90:10.

What is claimed is:

1. A method for isolating at least one target from a lignin source, the method comprising:
adding the lignin source to a base solution to form a suspension or solution;
applying energy to the suspension or solution;
adding an acid to the suspension or solution to precipitate lignin solids;
separating the lignin solids from the suspension or solution;
extracting the target from the suspension or solution with an organic solvent to form an organic solution comprising the target;
washing the organic solution with water and/or brine;
drying the organic solution; and
removing the organic solvent from the organic solution to recover a first product comprising a first quantity of the target.

2. The method according to claim 1, wherein the lignin source is selected from a sugar cane bagasse, a corn stover, and a combination thereof.

3. The method according to claim 1, wherein the lignin source is a powder.

4. The method according to claim 1, wherein the base solution is a sodium hydroxide solution.

5. The method according to claim 1, wherein the at least one target is selected from ferulic acid, p-coumaric acid, and a combination thereof.

6. The method according to claim 1, wherein applying energy to the suspension or solution comprises heating.

7. The method according to claim 6, wherein the suspension or solution is heated to a temperature above 100 degrees Celsius.

8. The method according to claim 5, wherein the heating is performed at a pressure greater than atmospheric pressure.

9. The method according to claim 8, wherein the suspension or solution is heated to a temperature of about 150° C. at a pressure of about 20 psi.

10. The method according to claim 1, wherein applying energy to the lignin source comprises sonicating the lignin source.

11. The method according to claim 10, wherein the sonicating is performed at room temperature.

12. The method according to claim 1, wherein applying energy to the lignin source comprises irradiating the lignin source with microwaves while maintaining a temperature below 90 degrees Celsius.

13. The method according to claim 1, wherein the organic solvent is ethyl acetate.

14. The method according to claim 1, further comprising adding an organic solvent to the lignin solids to form an organic solution comprising the lignin solids,
separating the lignin solids from the organic solution;
washing the organic solution with water and/or brine;
drying the organic solution; and
removing the organic solvent from the organic solution to recover a second product comprising a second quantity of the target.
